(19) United States
(12) Patent Application Publication
(10) Pub. No.: US 2025/0312421 A1
(43) Pub. Date: Oct. 9, 2025

(54) INJECTABLE COLLAGEN MATERIAL AND PREPARATION METHOD THEREOF

(71) Applicant: Hangzhou Junxing Biotechnology Co., Ltd, Hangzhou (CN)

(72) Inventor: Mingchun DU, Hangzhou (CN)

(21) Appl. No.: 19/242,469

(22) Filed: Jun. 18, 2025

Related U.S. Application Data
(63) Continuation of application No. PCT/CN2023/140491, filed on Dec. 21, 2023.

(30) Foreign Application Priority Data
Dec. 26, 2022 (CN) 202211673223.5

(51) Int. Cl. A61K 38/39; A61K 9/00

(52) U.S. Cl. CPC A61K 38/39; A61K 9/0019

(57) ABSTRACT

An injectable collagen material and a preparation method thereof. The injectable collagen material possesses characteristics such as pH neutrality, homogeneity, fluidity and the like, which meets the requirement that the aqueous collagen solution can be directly injected into the body and can form a collagen hydrogel with a certain mechanical strength in the body.

### TECHNICAL FIELD

The present application relates to a biomaterial and a preparation method thereof, in particular to an injectable collagen material and a preparation method thereof.

### BACKGROUND

Collagen, the main structural protein of connective tissues, is widely distributed in tissues and organs such as bone, skin, blood vessel, ligament, cartilage, muscle and tendon, accounting for 25% to 30% of the total protein. To date, more than 20 types of collagens have been identified, with type I collagen being the most abundant at about 90%. Due to its good biocompatibility, biodegradability and bioactivity, collagen is widely used as a conventional biomaterial in tissue engineering, regenerative medicine and other fields.

Injectable collagen materials, as one of the application forms of collagen, are typically obtained by dissolving salt-precipitated or solid collagen in an acidic solution followed by dialysis. During the dialysis process, the pH of the acidic aqueous collagen solution will gradually turn into neutral. Since the isoelectric point of collagen ranges from 7.5 to 7.8, collagen molecules dissolved in the aqueous solution tend to gradually self-assemble into collagen fibers. Therefore, conventional injectable collagen materials are essentially dispersions of phase-separated collagen fibers concentrated in an aqueous solution, i.e., a non-homogeneous aqueous suspension of collagen fibers rather than a homogeneous aqueous solution of fully dissolved collagen molecules. Such a collagen fiber suspension usually requires additional processing such as high-speed shearing or filtration to meet the injection requirements. Due to the essence of a non-homogeneous aqueous suspension of collagen fibers, the collagen fiber suspension will not form a collagen hydrogel with a certain mechanical strength after being injected into the body.

In view of the above problems in the preparation and use of injectable collagen materials, there is a need to develop an injectable collagen material with a neutral pH and homogeneous fluidity that can form a collagen hydrogel with a certain mechanical strength in the body, as well as a preparation method of an injectable collagen material.

### SUMMARY OF THE INVENTION

An object of the present application is to provide an injectable collagen material with a neutral pH and homogeneous fluidity that can form a collagen hydrogel with a certain mechanical strength in the body. Another object of the present application is to provide a preparation method of an injectable collagen material.

The preparation method of an injectable collagen material provided in the present application includes the following steps in order:
(1) dissolving a solid collagen material in water fully, centrifuging, and collecting a supernatant; the solid collagen material is selected from one or a combination of two or more of type I, type II, type III, and type V collagen; a total concentration of various types of collagens in the supernatant is greater than or equal to 0.1 wt. % but less than or equal to 5 wt. %;
(2) adding a simulated body fluid component into the supernatant obtained in the step (1); the simulated body fluid component includes one or a combination of two or more of Na⁺, K⁺, Mg²⁺, Ca²⁺, Cl⁻, HCO₃⁻, HPO₄²⁻, SO₄²⁻, CO₃²⁻, PO₄³⁻, and H₂PO₄⁻;
(3) centrifuging the solution obtained in the step (2), and collecting a supernatant;
(4) adjusting a pH value of the supernatant obtained in the step (3) with an alkaline solution and an acidic solution to a final range of greater than or equal to 6.0 but less than or equal to 8.0;
(5) centrifuging the solution obtained in the step (4), and collecting a supernatant;
(6) adding a protective molecule into the supernatant obtained in the step (5); the protective molecule includes at least one of sodium dodecyl sulfate, sodium dodecyl benzene sulfonate, Tween, Triton, ethylene glycol, phenylalanine, proline, lecithin, glutamic acid, lysine, cysteine, mangiferin, glycine, aspartic acid, n-butanol, propylene glycol, a sodium salt of ethylenediamine tetraacetic acid, stearic acid, Span, glycerin, and gelatin;
(7) irradiating the solution obtained in the step (6) at low temperature;
(8) centrifuging the solution obtained in the step (7), and collecting a supernatant.

In the preparation method of an injectable collagen material provided in the present application, the rotational speed for the centrifugation in the step (3), step (5), and step (8) is 1000 to 10000 rpm. Due to the unavoidable phenomenon of collagen molecules self-assembling to form collagen fibers during the preparation process, in order to meet the injectability requirement of the material, centrifugation can be used to remove insoluble substances from the aqueous collagen solution, and the appropriate centrifugal speed can be selected based on the size of the injection needle. For example, a centrifugal speed of 1,000 rpm for an 8 G needle can meet the requirement of injection, and a centrifugal speed of 3,000 rpm for a 21 G needle can meet the requirement of injection.

In the preparation method of an injectable collagen material provided in the present application, the acidic solution in the step (4) includes at least one of salicylic acid, lactic acid, sulfuric acid, tartaric acid, citric acid, phosphoric acid, oxalic acid, acetic acid, ethanedioic acid, succinic acid, hydrochloric acid, maleic acid, benzoic acid, nitric acid, malic acid, nicotinic acid, sodium dihydrogen phosphate, and formic acid; and the alkaline solution includes at least one of tetramethyl ethylenediamine, sodium hydroxide, triethylamine, sodium carbonate, potassium hydroxide, calcium hydroxide, magnesium hydroxide, ammonia water, disodium hydrogen phosphate, and sodium bicarbonate. The selection of a suitable acidic solution or alkaline solution is primarily to adjust the pH value of the aqueous collagen solution to a range permissible for in vivo injection. Since the normal pH value of the human body is 7.35-7.45, collagen molecules within this range can easily self-assemble to form insoluble substances to clog the injection needle. Therefore, the acidic solution or alkaline solution tends to be a weak acid or weak base, which can interact synergistically with the simulated body fluid component and protective molecule added to the aqueous collagen solution, reducing the self-assembly behavior of collagen molecules and facilitating the use of the aqueous collagen solution in the form of injections.

In the preparation method of an injectable collagen material provided in the present application, the pH value of the supernatant in the step (4) has a final range of greater than or equal to 6.8 but less than or equal to 7.8. The pH value of the aqueous collagen solution is adjusted to be around the normal pH range of the human body to ensure good tissue compatibility when the material is injected into the body.

Compared with the prior art, the preparation method of an injectable collagen material provided in the present application involves steps of firstly selecting a suitable collagen type and controlling the total concentration of collagen, and secondly adding a simulated body fluid component, adjusting the pH value with an alkaline solution or an acidic solution and adding a protective molecule, which are synergistic with each other, and centrifuging several times during the process in order to remove insoluble substances from the aqueous collagen solution, ensuring that the aqueous collagen solution maintains homogeneous fluidity, collagen molecules do not aggregate to clog the needle, and even after irradiation, collagen molecules will not crosslink to clog the needle. An injectable collagen material with a neutral pH and homogeneous fluidity can be obtained by the preparation method, which can form a collagen hydrogel with a certain mechanical strength in the body after injection.

The present application provides an injectable collagen material which is obtained by the above preparation method of an injectable collagen material.

For the injectable collagen material of the present application, the injectable collagen material can be injected into the body with an 8-34 G needle; preferably, the injectable collagen material can be injected into the body with a 21-32 G needle; preferably, the injectable collagen material can be injected into the body with a 25-30 G needle. Due to differences in application scenarios, the use of the injectable collagen material of the present application requires the selection of different specifications of needles. For example, a 10 G needle with an inner diameter of about 2.69 mm can be selected for body cavity filling and a 32 G needle with an inner diameter of about 0.11 mm can be selected for facial micro plastic injection. Therefore, it is necessary to regulate the key parameters in the preparation method to ensure the homogeneous fluidity of the aqueous collagen solution without the problem of needle clogging.

The injectable collagen material of the present application can from a collagen hydrogel after being injected into the body, and the collagen hydrogel has a compression modulus ranging from 0.1 to 20 kPa. Since the collagen molecules in the injectable collagen material of the present application have not self-assembled to form collagen fibers before injection and have not been irradiated to undergo cross-linking reactions, after injection, a collagen hydrogel with a certain mechanical strength can be formed through the aggregation of collagen molecules and other interactions under the temperature conditions in the body, unlike a non-homogeneous aqueous suspension of collagen fibers in which collagen molecules have already aggregated before injection, preventing the formation of a collagen hydrogel with a certain mechanical strength after being injected into the body. The mechanical strength of the formed collagen hydrogel can be regulated by controlling the collagen concentration in the injectable collagen material.

The injectable collagen material of the present application is different from the non-homogeneous aqueous suspension of collagen fibers of conventional injectable collagen materials in that it is prepared by the above preparation method of an injectable collagen material, is pH-neutral, can flow homogeneously, and can form a collagen hydrogel with a certain mechanical strength in the body.

### BRIEF DESCRIPTION OF THE DRAWINGS

FIG. 1 is an image showing the turbidity test results of injectable collagen materials (FIG. 1A shows the sample of Example 1, FIG. 1B shows the sample of Example 2, FIG. 1C shows the sample of Example 3, FIG. 1D shows the sample of Comparative Example 1, and FIG. 1E shows the sample of Comparative Example 2);

FIG. 2 shows the needles of different specifications used for testing the injectable collagen materials;

FIG. 3 shows the subcutaneous injection test of injectable collagen materials in nude mice.

### DETAILED DESCRIPTION

### Example 1

A preparation method of an injectable collagen material, including the following steps in order:
(1) 1 g of type I collagen was fully dissolved in 1000 g of water and centrifuged at a rotational speed of 2000 rpm, and the supernatant was collected;
(2) 2 g of KHCO₃, 0.01 g of CaCl₂, and 0.5 g of NaH₂PO₄ were added into the supernatant obtained in the step (1);
(3) the solution obtained in the step (2) was centrifuged at a rotational speed of 1000 rpm and the supernatant was collected;
(4) the supernatant obtained in the step (3) was adjusted to pH 7.2 with 1 M succinic acid, 0.5 M acetic acid, and 2 M sodium carbonate;
(5) the solution obtained in the step (4) was centrifuged at a rotational speed of 3000 rpm and the supernatant was collected;
(6) 1% Triton, 0.5% a sodium salt of ethylenediamine tetraacetic acid, and 0.2% glycine were added into the supernatant obtained in the step (5);
(7) the solution obtained in step (6) was subjected to Co⁶⁰ irradiation at 0° C.;
(8) the solution obtained in the step (7) was centrifuged at a rotational speed of 3000 rpm and the supernatant was collected.

The injectable collagen material prepared in this example can be formed into an injectable collagen material with homogeneous fluidity, in which the collagen molecules are fully dissolved in an aqueous solution in a molecular state, so that the collagen material can be injected through a 32 G needle with an inner diameter of 0.11 mm and can form a collagen hydrogel with a compression modulus of 0.25±0.08 KPa after being injected into the body.

### Example 2

A preparation method of an injectable collagen material, including the following steps in order:
(1) 2 g of type II collagen and 0.1 g of type III collagen were fully dissolved in 200 g of water and centrifuged at a rotational speed of 3000 rpm, and the supernatant was collected;
(2) 0.1 g of NaCl, 0.1 g of Na₂CO₃, and 0.1 g of K₃PO₄ were added into the supernatant obtained in the step (1);
(3) the solution obtained in the step (2) was centrifuged at a rotational speed of 2000 rpm and the supernatant was collected;
(4) the supernatant obtained in the step (3) was adjusted to pH 6.0 with 0.5 M phosphoric acid and 0.1 M potassium hydroxide;
(5) the solution obtained in the step (4) was centrifuged at a rotational speed of 4000 rpm and the supernatant was collected;
(6) 0.4% n-butanol, 1% aspartic acid, and 2% glycerin were added into the supernatant obtained in the step (5);
(7) the solution obtained in step (6) was subjected to electron beam irradiation at 4° C.;
(8) the solution obtained in the step (7) was centrifuged at a rotational speed of 5000 rpm and the supernatant was collected.

The injectable collagen material prepared in this example can be formed into a homogeneous flowable injectable collagen material, in which the collagen molecules are fully dissolved in an aqueous solution in a molecular state that can be injected through a 30 G needle with an inner diameter of 0.16 mm and can form a collagen hydrogel with a compression modulus of 5.92±0.43 KPa after injection into the body.

### Example 3

A preparation method of an injectable collagen material, including the following steps in order:
(1) 40 g of type I collagen and 0.1 g of type V collagen were fully dissolved in 500 g of water and centrifuged at a rotational speed of 6000 rpm, and the supernatant was collected;
(2) 0.5 g of K₂SO₄, 1 g of KH₂PO₄, and 2 g of K₂CO₃ were added into the supernatant obtained in the step (1);
(3) the solution obtained in the step (2) was centrifuged at a rotational speed of 4000 rpm and the supernatant was collected;
(4) the supernatant obtained in the step (3) was adjusted to pH 8.0 with 2 M salicylic acid, 1 M triethylamine, and 1 M ammonia water;
(5) the solution obtained in the step (4) was centrifuged at a rotational speed of 6000 rpm and the supernatant was collected;
(6) 0.01% gelatin, 0.2% ethylene glycol, and 1% proline were added into the supernatant obtained in the step (5);
(7) the solution obtained in step (6) was subjected to gamma-ray irradiation at 4° C.;
(8) the solution obtained in the step (7) was centrifuged at a rotational speed of 10000 rpm and the supernatant was collected.

The injectable collagen material prepared in this example can be formed into a homogeneous flowable injectable collagen material, in which the collagen molecules are fully dissolved in an aqueous solution in a molecular state that can be injected through a 22 G needle with an inner diameter of 0.41 mm and can form a collagen hydrogel with a compression modulus of 17.86±0.15 KPa after injection into the body.

In the preparation method of an injectable collagen material provided in the present application, the solid collagen material in the step (1) is selected from one or a combination of two or more of type I, type II, type III, and type V collagen, and the selection of the solid collagen material is not limited to the types disclosed in Examples 1-3, other types can also be used, for example, type II, type III or type V can be used alone, or a combination of four types I, II, III, and V can be used. The mass of the solid collagen material can also be adjusted, as long as the total concentration of various types of collagens in the supernatant of step (1) is greater than or equal to 0.1 wt. % but less than or equal to 5 wt. %. The reason for choosing such a total collagen concentration is as follows: if the collagen concentration is less than 0.1 wt. %, a collagen hydrogel with a certain mechanical strength cannot be formed in the body after injection, and if the collagen concentration is greater than 5 wt. %, the aqueous collagen solution would be too viscous to be injected. The selection of the total collagen concentration range is a prerequisite for the prepared injectable collagen material to meet the requirements for injection and use.

The simulated body fluid component in the step (2) of the present application includes one or a combination of two or more of Na⁺, K⁺, Mg²⁺, Ca²⁺, Cl⁻, HCO₃⁻, HPO₄²⁻, SO₄²⁻, CO₃²⁻, PO₄³⁻, and H₂PO₄⁻. Since the isoelectric point of collagen ranges from 7.5 to 7.8, collagen molecules in the aqueous collagen solution tend to self-assemble into collagen fibers during the subsequent pH adjustment process, which may easily clog the injection needle, making injection impossible. The addition of the simulated body fluid component, on one hand, balances the ionic strength in the aqueous solution to weaken the interaction between collagen molecules, and on the other hand, simulates the in vivo environment to enhance the tissue compatibility of the injectable collagen materials.

In the step (4) of the present application, the acidic solution includes at least one of salicylic acid, lactic acid, sulfuric acid, tartaric acid, citric acid, phosphoric acid, oxalic acid, acetic acid, ethanedioic acid, succinic acid, hydrochloric acid, maleic acid, benzoic acid, nitric acid, malic acid, nicotinic acid, sodium dihydrogen phosphate, and formic acid; and the alkaline solution includes at least one of tetramethyl ethylenediamine, sodium hydroxide, triethylamine, sodium carbonate, potassium hydroxide, calcium hydroxide, magnesium hydroxide, ammonia water, disodium hydrogen phosphate, and sodium bicarbonate. The selection of a suitable acidic solution or alkaline solution is primarily to adjust the pH value of the aqueous collagen solution to a range permissible for in vivo injection. Since the normal pH value of the human body is 7.35-7.45, collagen molecules within this range can easily self-assemble to form insoluble substances to clog the injection needle. Therefore, the acidic solution or alkaline solution tends to be a weak acid or weak base, which can interact synergistically with the simulated body fluid component and protective molecule added to the aqueous collagen solution, reducing the self-assembly behavior of collagen molecules and facilitating the use of the aqueous collagen solution in the form of injections. The pH value of the supernatant in the step (4) has a final range of greater than or equal to 6.0 but less than or equal to 8.0; preferably, the pH value of the supernatant in the step (4) has a final range of greater than or equal to 6.8 but less than or equal to 7.8. The pH value of the aqueous collagen solution is adjusted to be around the normal pH range of the human body to ensure good tissue compatibility when the material is injected into the body.

The protective molecule in the step (6) of the present application includes at least one of sodium dodecyl sulfate, sodium dodecyl benzene sulfonate, Tween, Triton, ethylene glycol, phenylalanine, proline, lecithin, glutamic acid, lysine, cysteine, mangiferin, glycine, aspartic acid, n-butanol, propylene glycol, a sodium salt of ethylenediamine tetraacetic acid, stearic acid, Span, glycerin, and gelatin. The addition of a suitable protective molecule is mainly to have weak interactions such as hydrogen bonds with the collagen molecules in the aqueous solution, thereby reducing the possibility of needle clogging caused by cross-linking and other reactions of the collagen molecules during the irradiation process. Meanwhile, the protective molecule also weakens the interaction between the collagen molecules in the aqueous solution, thereby reducing the possibility of the self-assembly of collagen molecules to form insoluble substances that could clog the needle.

The rotational speed for the centrifugation in the step (3), step (5), and step (8) of the present application is 1000 to 10000 rpm. Due to the unavoidable phenomenon of collagen molecules self-assembling to form collagen fibers during the preparation process, in order to meet the injectability requirement of the material, centrifugation can be used to remove insoluble substances from the aqueous collagen solution, and the appropriate centrifugal speed can be selected based on the size of the injection needle. For example, a centrifugal speed of 1,000 rpm for an 8 G needle can meet the requirement of injection, and a centrifugal speed of 3,000 rpm for a 21 G needle can meet the requirement of injection.

The present application provides an injectable collagen material which is obtained by the above preparation method of an injectable collagen material.

For the injectable collagen material of the present application, the injectable collagen material can be injected into the body with an 8-34 G needle; preferably, the injectable collagen material can be injected into the body with a 21-32 G needle; preferably, the injectable collagen material can be injected into the body with a 25-30 G needle. Due to differences in application scenarios, the use of the injectable collagen material of the present application requires the selection of different specifications of needles. For example, a 10 G needle with an inner diameter of about 2.69 mm can be selected for body cavity filling and a 32 G needle with an inner diameter of about 0.11 mm can be selected for facial micro plastic injection. Therefore, it is necessary to regulate the key parameters in the preparation method to ensure the homogeneous fluidity of the aqueous collagen solution without the problem of needle clogging.

The injectable collagen material of the present application can from a collagen hydrogel after being injected into the body, and the collagen hydrogel has a compression modulus ranging from 0.1 to 20 kPa. Since the collagen molecules in the injectable collagen material of the present application have not self-assembled to form collagen fibers before injection and have not been irradiated to undergo cross-linking reactions, after injection, a collagen hydrogel with a certain mechanical strength can be formed through the aggregation of collagen molecules and other interactions under the temperature conditions in the body, unlike a non-homogeneous aqueous suspension of collagen fibers in which collagen molecules have already aggregated before injection, preventing the formation of a collagen hydrogel with a certain mechanical strength after being injected into the body. The mechanical strength of the formed collagen hydrogel can be regulated by controlling the collagen concentration in the injectable collagen material.

Different from the non-homogeneous aqueous suspension of collagen fibers of conventional injectable collagen materials, the injectable collagen material of the present application provides an injectable collagen material with a neutral pH and homogeneous fluidity that can form a collagen hydrogel with a certain mechanical strength in the body. In the preparation method of the present application, the steps of adding a simulated body fluid component, adjusting the pH value with an alkaline solution or acidic solution, and adding a protective molecule act synergistically, ensuring that the aqueous collagen solution maintains homogeneous fluidity, collagen molecules do not aggregate to clog the needle, and even after irradiation, collagen molecules will not crosslink to clog the needle. The injectable collagen material of the present application can be injected into the body in a homogeneous flowing state to form a collagen hydrogel with a certain mechanical strength.

The effects of the present application will be illustrated below by comparing Examples 1-3 with Comparative Examples 1 and 2.

### Comparative Test on the Effects of Injectable Collagen Materials

### I. Preparation of Comparative Collagen Materials

Comparative Example 1: A preparation method of a collagen material, including the following steps in order:
(1) 1 g of type I collagen was fully dissolved in 1000 g of water;
(2) 2 g of KHCO₃, 0.01 g of CaCl₂, and 0.5 g of NaH₂PO₄ were added into the supernatant obtained in the step (1);
(3) 1% Triton and 0.2% glycine were added into the supernatant obtained in the step (2);
(4) the solution obtained in the step (3) was subjected to Co⁶⁰ irradiation at room temperature.

Comparative Example 2: A preparation method of a collagen material, including the following steps in order:
(1) 1 g of type I collagen was fully dissolved in 1000 g of water and centrifuged at a rotational speed of 2000 rpm, and the supernatant was then collected;
(2) the supernatant obtained in the step (1) was adjusted to pH 7.2 with 1 M succinic acid and 2 M sodium carbonate;
(3) the solution obtained in the step (2) was centrifuged at a rotational speed of 3000 rpm and the supernatant was collected;
(4) the solution obtained in step (3) was subjected to Co⁶⁰ irradiation at 0° C.;
(5) the solution obtained in the step (4) was centrifuged at a rotational speed of 3000 rpm and the supernatant was collected.

### II. Effect Test Comparison

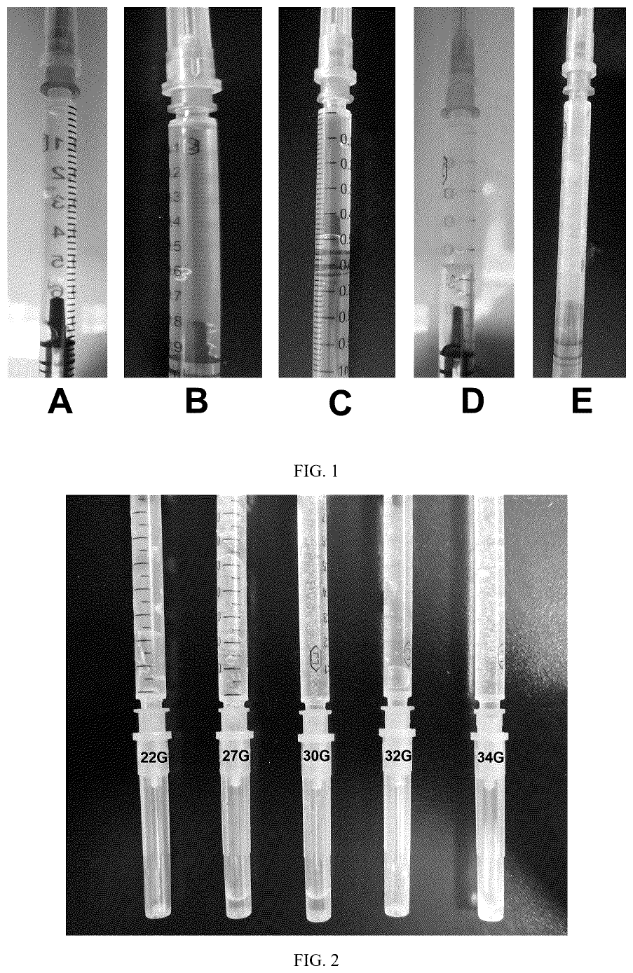

(1) Turbidity test of injectable collagen materials: The turbidity of injectable collagen materials was observed on a turbidimeter (TZD-BZ-905, Suzhou DiagVita Technology Co., Ltd.) at a wavelength of 625 nm. The test results are shown in Table 1. The image showing the turbidity test results of injectable collagen materials is shown in FIG. 1, where, FIG. 1A shows the sample of Example 1, FIG. 1B shows the sample of Example 2, FIG. 1C shows the sample of Example 3, FIG. 1D shows the sample of Comparative Example 1, and FIG. 1E shows the sample of Comparative Example 2.

TABLE 1
Turbidity test of injectable collagen materials

| | | | | Comparative | Comparative |
|---|---|---|---|---|---|
| Samples | Example 1 | Example 2 | Example 3 | Example 1 | Example 2 |
| Turbidity (×10⁴ | 0.36 ± 0.08 | 0.23 ± 0.02 | 0.39 ± 0.06 | 4.15 ± 0.18 | 6.21 ± 0.32 |
| NTU) | | | | | |

As can be seen from Table 1, in the injectable collagen materials of Examples 1-3, the collagen molecules remain in a molecular state and are fully dissolved in the aqueous solution without aggregating to form collagen fibers, and the aqueous solution has homogeneous fluidity and low turbidity. In FIGS. 1A, 1B and 1C, the injectable collagen materials of Examples 1, 2 and 3 are relatively transparent, and the scale of the syringe can be clearly observed. In contrast, in Comparative Examples 1-2, the collagen molecules have aggregated to form collagen fibers, forming a collagen fiber suspension similar to conventional injectable collagen materials, and the turbidity of the aqueous solution is relatively high. In FIGS. 1D and 1E, the aqueous solutions of Comparative Examples 1 and 2 are relatively turbid, and the scale of the syringe cannot be clearly observed.

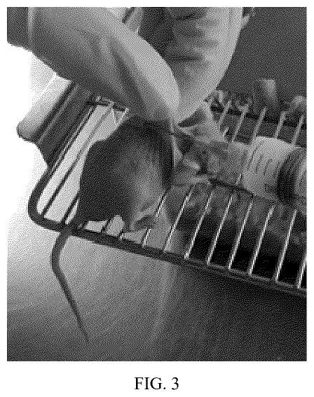

(2) Injection test of injectable collagen materials through needles of different specifications: The injectable collagen material prepared in Example 1 was subjected to injection tests using needles of different specifications as shown in FIG. 2. The figure shows, from left to right in order, a 22 G needle with an inner diameter of 0.41 mm, a 27 G needle with an inner diameter of 0.21 mm, a 30 G needle with an inner diameter of 0.16 mm, a 32 G needle with an inner diameter of 0.11 mm, and a 34 G needle with an inner diameter of 0.06 mm. As can be seen from FIG. 2, the injectable collagen material prepared in Example 1 has homogeneous fluidity, and the collagen molecules do not aggregate to form collagen fibers. Therefore, the injection operation can be performed through a needle of the above specifications without clogging the needle. However, in Comparative Example 1, a collagen fiber suspension is formed, which is a non-homogeneous system. The collagen fibers are very likely to aggregate and clog the needle, making it impossible to inject through a needle of the above specifications.

(3) Subcutaneous injection test of injectable collagen material in nude mice: The injectable collagen material in Example 1 was subjected to subcutaneous injection test in nude mice through an 8 G needle. As can be seen from FIG. 3, the material can be easily injected, meeting the injectability requirement.

(4) Compression modulus test of hydrogel formed in vivo from injectable collagen materials: the samples of Examples 1-3 and Comparative Examples 1-2 were injected subcutaneously into the mice. After 24 hours, the injected materials were separated from the mouse tissues and subjected to pressure test. A uniaxial mechanical tester (E10000, Instron) was used, with a load of 1 KN, a compression speed of 0.4 mm min⁻¹, and a temperature of 25° C. During the test, the samples were kept in a moist state. The sample size was 5 mm×5 mm×5 mm, and the compression amount was 30%. The compression modulus was calculated from the slope of the stress-strain curve. The test results are shown in Table 2.

TABLE 2
Compression modulus test for hydrogel formed from injectable collagen materials

| | | | | Comparative | Comparative |
|---|---|---|---|---|---|
| | Example 1 | Example 2 | Example 3 | Example 1 | Example 2 |
| Compression | 0.25 ± 0.08 | 5.92 ± 0.43 | 17.86 ± 0.15 | None | None |
| modulus (KPa) | | | | | |

As can be seen from Table 2, the injectable collagen materials of Examples 1-3 maintain a homogeneous flowing aqueous solution state and can form a collagen hydrogel with a certain mechanical strength in mice. In contrast, the aqueous collagen solutions in Comparative Examples 1-2 are actually collagen fiber suspensions, which cannot form a collagen hydrogel in mice and are in an amorphous state, so their compression modulus cannot be tested.

Based on the above tests, it can be seen that compared with Comparative Examples 1-2, injectable collagen materials with homogeneous fluidity can be formed in Examples 1-3, in which the collagen molecules are fully dissolved in the aqueous solution in a molecular state, allowing injection operations through needles with smaller inner diameters. After being injected into the body, a collagen hydrogel with a certain mechanical strength can be formed. In contrast, in Comparative Examples 1-2, collagen fiber suspensions are formed, in which the collagen molecules have aggregated to form collagen fibers. The suspensions are not homogeneous and cannot form a collagen hydrogel after being injected into the body.

The above examples merely describe the preferred embodiments of the present application. Without departing from the design spirit of the present application, various modifications and improvements made by those of ordinary skill in the art to the technical solutions of the present application should all fall within the protection scope determined by the claims of the present application.

### INDUSTRIAL APPLICABILITY

The preparation method of a collagen material provided by the present application can be used to prepare an injectable collagen material. As one of the application forms of collagen, the collagen material prepared by the present application is an injectable collagen material with a neutral pH and homogeneous fluidity that can form a collagen hydrogel with a certain mechanical strength in the body, and can be widely used in tissue engineering, regenerative medicine and other fields.

1. A preparation method of an injectable collagen material, comprising the following steps in order:
(1) dissolving a solid collagen material in water fully, centrifuging, and collecting a supernatant; the solid collagen material is selected from one or a combination of two or more of type I, type II, type III, and type V collagen; a total concentration of various types of collagens in the supernatant is greater than or equal to 0.1 wt. % but less than or equal to 5 wt. %;
(2) adding a simulated body fluid component into the supernatant obtained in the step (1); the simulated body fluid component comprises one or a combination of two or more of Na⁺, K⁺, Mg²⁺, Ca²⁺, Cl⁻, HCO₃⁻, HPO₄²⁻, SO₄²⁻, CO₃²⁻, PO₄³⁻, and H₂PO₄⁻;
(3) centrifuging the solution obtained in the step (2), and collecting a supernatant;
(4) adjusting a pH value of the supernatant obtained in the step (3) with an alkaline solution and an acidic solution to a final range of greater than or equal to 6.0 but less than or equal to 8.0;
(5) centrifuging the solution obtained in the step (4), and collecting a supernatant;
(6) adding a protective molecule into the supernatant obtained in the step (5); the protective molecule comprises at least one of sodium dodecyl sulfate, sodium dodecyl benzene sulfonate, Tween, Triton, ethylene glycol, phenylalanine, proline, lecithin, glutamic acid, lysine, cysteine, mangiferin, glycine, aspartic acid, n-butanol, propylene glycol, a sodium salt of ethylenediamine tetraacetic acid, stearic acid, Span, glycerin, and gelatin;
(7) irradiating the solution obtained in the step (6) at low temperature;
(8) centrifuging the solution obtained in the step (7), and collecting a supernatant.

2. The preparation method of an injectable collagen material according to claim 1, wherein, a rotational speed for the centrifugation in the step (3) is 1000 to 10000 rpm.

3. The preparation method of an injectable collagen material according to claim 1, wherein, a rotational speed for the centrifugation in the step (5) is 1000 to 10000 rpm.

4. The preparation method of an injectable collagen material according to claim 1, wherein, a rotational speed for the centrifugation in the step (8) is 1000 to 10000 rpm.

5. The preparation method of an injectable collagen material according to claim 1, wherein, the acidic solution in the step (4) comprises at least one of salicylic acid, lactic acid, sulfuric acid, tartaric acid, citric acid, phosphoric acid, oxalic acid, acetic acid, ethanedioic acid, succinic acid, hydrochloric acid, maleic acid, benzoic acid, nitric acid, malic acid, nicotinic acid, sodium dihydrogen phosphate, and formic acid; and the alkaline solution comprises at least one of tetramethyl ethylenediamine, sodium hydroxide, triethylamine, sodium carbonate, potassium hydroxide, calcium hydroxide, magnesium hydroxide, ammonia water, disodium hydrogen phosphate, and sodium bicarbonate.

6. The preparation method of an injectable collagen material according to claim 1, wherein, the pH value of the supernatant in the step (4) has a final range of greater than or equal to 6.8 but less than or equal to 7.8.

7. An injectable collagen material prepared by using the preparation method of an injectable collagen material according to claim 1.

8. The injectable collagen material according to claim 7, wherein, the injectable collagen material can be injected into the body with an 8-34 G needle; preferably, the injectable collagen material can be injected into the body with a 21-32 G needle; preferably, the injectable collagen material can be injected into the body with a 25-30 G needle.

9. The injectable collagen material according to claim 8, wherein, the injectable collagen material can from a collagen hydrogel after being injected into body, and the collagen hydrogel has a compression modulus ranging from 0.1 to 20 kPa.
